(19) United States
(12) Patent Application Publication
(10) Pub. No.: US 2010/0015061 A1
(43) Pub. Date: Jan. 21, 2010

(54) Pharmaceutical Compositions Based on Anticholinergics and Andolast

(75) Inventor: Michael P. Pieper, Biberach (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: Boehringer Ingeheim International GmnH, Ingelheim (DE)

(21) Appl. No.: 12/159,814

(22) PCT Filed: Dec. 22, 2006
(86) PCT No.: PCT/EP2006/070137

§ 371 Date: Oct. 27, 2008

(30) Foreign Application Priority Data
Jan. 6, 2006 (EP) 06100127.7

(51) Int. Cl. A61K 9/12; A61K 31/439; A61P 11/00

(52) U.S. Cl. 424/45; 514/305

(57) ABSTRACT

The present invention relates to novel pharmaceutical compositions based on anticholinergics and andolast, processes for preparing them and their use in the treatment of respiratory diseases.

The present invention relates to novel pharmaceutical compositions based on anticholinergics and andolast, processes for preparing them and their use in the treatment of respiratory diseases.

### DESCRIPTION OF THE INVENTION

The present invention relates to novel pharmaceutical compositions based on anticholinergics 1 and andolast 2, processes for preparing them and their use in the treatment of respiratory diseases.

Surprisingly, an unexpectedly beneficial therapeutic effect can be observed in the treatment of diseases of the respiratory tract if one or more anticholinergics are used together with andolast.

The effects mentioned above may be observed both when the two active substances are administered simultaneously in a single active substance formulation and when they are administered successively in separate formulations. According to the invention, it is preferable to administer the active substance ingredients simultaneously in a single formulation.

By the anticholinergics 1 which may be used within the scope of the present invention are meant the compounds which contain, in addition to tiotropium, oxitropium, flutropium, ipratropium, glycopyrronium as counter-ion (anion) fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably, chloride, bromide, iodide, sulphate, methanesulphonate or para-toluenesulphonate. Within the scope of the present invention, the methanesulphonate, chloride, bromide and iodide are preferred of all the salts 1, the methanesulphonate and bromide being of particular importance. Of outstanding importance according to the invention are salts 1 selected from among tiotropium bromide, oxitropium bromide, ipratropium bromide and glycopyrronium bromide. Tiotropium bromide is particularly preferred, preferably in the form of its crystalline monohydrate as disclosed in WO 02/30928 A1.

In another preferred embodiment the anticholinergic 1 is selected from the group of compounds consisting of tropenol 2,2-diphenylpropionic acid ester methobromide, scopine 2,2-diphenylpropionic acid ester methobromide, scopine 2-fluoro-2,2-diphenylacetic acid ester methobromide, tropenol 2-fluoro-2,2-diphenylacetic acid ester methobromide, tropenol 3,3′,4,4′-tetrafluorobenzilic acid ester methobromide, scopine 3,3′,4,4′-tetrafluorobenzilic acid ester methobromide, scopine 4,4′-difluorobenzilic acid ester methobromide, tropenol 4,4′-difluorobenzilic acid ester methobromide, scopine 3,3′-difluorobenzilic acid ester methobromide, tropenol 3,3′-difluorobenzilic acid ester methobromide, tropenol 9-hydroxy-fluorene-9-carboxylate methobromide, tropenol 9-fluoro-fluorene-9-carboxylate methobromide, scopine 9-hydroxy-fluorene-9-carboxylate methobromide, scopine 9-fluoro-fluorene-9-carboxylate methobromide, tropenol 9-methyl-fluorene-9-carboxylate methobromide, scopine 9-methyl-fluorene-9-carboxylate methobromide, cyclopropyltropine benzilate methobromide, cyclopropyltropine 2,2-diphenylpropionate methobromide, cyclopropyltropine 9-hydroxy-xanthene-9-carboxylate methobromide, cyclopropyltropine 9-methyl-fluorene-9-carboxylate methobromide, cyclopropyltropine 9-methyl-xanthene-9-carboxylate methobromide, cyclopropyltropine 9-hydroxy-fluorene-9-carboxylate methobromide, cyclopropyltropine methyl 4,4′-difluorobenzilate methobromide, tropenol 9-hydroxy-xanthene-9-carboxylate methobromide, scopine 9-hydroxy-xanthene-9-carboxylate methobromide, tropenol 9-methyl-xanthene-9-carboxylate methobromide, scopine 9-methyl-xanthene-9-carboxylate methobromide, tropenol 9-ethyl-xanthene-9-carboxylate methobromide, tropenol 9-difluoromethyl-xanthene-9-carboxylate methobromide, and scopine 9-hydroxymethyl-xanthene-9-carboxylate methobromide. In the alternative to the methobromides mentioned hereinbefore, the anticholinergic may be selected from the corresponding fluorides, chlorides, iodides, sulphates, phosphates, methanesulphonates, nitrates, maleates, acetates, citrates, fumarates, tartrates, oxalates, succinates, benzoates and p-toluenesulphonates, preferably from the corresponding chlorides, iodides, sulphates, methanesulphonate or para-toluenesulphonates.

In another preferred embodiment the anticholinergic 1 is selected from the group of compounds consisting of tropenol 2,2-diphenylpropionic acid ester methobromide, scopine 2,2-diphenylpropionic acid ester methobromide, tropenol 9-hydroxy-fluorene-9-carboxylate methobromide, tropenol 9-fluoro-fluorene-9-carboxylate methobromide, scopine 9-hydroxy-fluorene-9-carboxylate methobromide, scopine 9-fluoro-fluorene-9-carboxylate methobromide, tropenol 9-methyl-fluorene-9-carboxylate methobromide, and scopine 9-methyl-fluorene-9-carboxylate methobromide. In the alternative to the methobromides mentioned hereinbefore, the anticholinergic may be selected from the corresponding fluorides, chlorides, iodides, sulphates, phosphates, methanesulphonates, nitrates, maleates, acetates, citrates, fumarates, tartrates, oxalates, succinates, benzoates and p-toluenesulphonates, preferably from the corresponding chlorides, iodides, sulphates, methanesulphonate or para-toluenesulphonates.

In another preferred embodiment the anticholinergic 1 is selected from the compounds of formula 1a

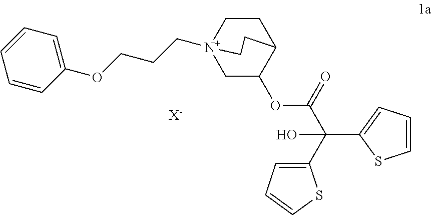

wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
   optionally in the form of the racemates, enantiomers or hydrates thereof.

Preferred medicament combinations contain salts of formula 1a, wherein
X⁻ denotes an anion with a single negative charge, preferably an anion selected from among the fluoride, chloride, bromide, methanesulphonate and p-toluenesulphonate, preferably bromide,
   optionally in the form of the racemates, enantiomers or hydrates thereof.

Preferred medicament combinations contain salts of formula 1a, wherein
X⁻ denotes an anion with a single negative charge, preferably an anion selected from among the chloride, bromide and methanesulphonate, preferably bromide,
   optionally in the form of the racemates, enantiomers or hydrates thereof.

Particularly preferred medicament combinations contain the compound of formula 1a in the form of the bromide.

Of particular importance are those medicament combinations which contain the enantiomers of formula 1a-en

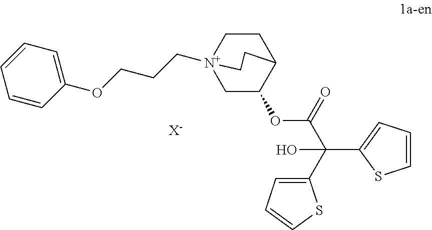

wherein X⁻ may have the above-mentioned meanings.

All of the compounds 1 mentioned hereinbefore are salts consisting of a pharmacologically active cation in combination with an anion. Within the instant application any reference to compounds 1′ is to be understood as a reference to the pharmacologically active cation.

Andolast 2 is N-4-(1H-tetrazol-5-yl)phenyl-4-(1H-tetrazol-5-yl)benzamide disodium salt, also known in the art as CR 2039. Pharmaceutically acceptable salts of andolast include but are not limited to alkali metal or earth alkali metal salts, as lithium, potassium, magnesium and the like.

The compounds 1 may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention. Further included are all mixtures of the enantiomers. Also included within the scope of the invention are polymorphs and hydrates of the compounds of the instant invention.

The term “therapeutically effective amount” shall mean that amount of a drug or pharmaceutical agent that will elicit the biological or medical response of a tissue, system, animal or human that is being sought by a researcher or clinician.

The pharmaceutical combinations of 1 and 2 according to the invention are preferably administered by inhalation. Suitable inhalable powders packed into suitable capsules (inhalettes) may be administered using suitable powder inhalers. Alternatively, the drug may be inhaled by the application of suitable inhalation aerosols. These also include inhalation aerosols which contain HFA134a, HFA227 or a mixture thereof as propellant gas, for example. The drug may also be inhaled using suitable solutions of the pharmaceutical combination consisting of 1 and 2. In one aspect, therefore, the invention relates to a pharmaceutical composition which contains a combination of 1 and 2.

In another aspect the present invention relates to a pharmaceutical composition which contains one or more salts 1 and 2, optionally in the form of their solvates or hydrates. The active substances may be combined in a single preparation or contained in two separate formulations. Pharmaceutical compositions which contain the active substances 1 and 2 in a single preparation are preferred according to the invention.

In another aspect the present invention relates to a pharmaceutical composition which contains, in addition to therapeutically effective quantities of 1 and 2, a pharmaceutically acceptable excipient. In another aspect the present invention relates to a pharmaceutical composition which does not contain any pharmaceutically acceptable excipient in addition to therapeutically effective quantities of 1 and 2.

The present invention also relates to the use of 1 and 2 for preparing a pharmaceutical composition containing therapeutically effective quantities of 1 and 2 for inflammatory and/or obstructive diseases of the respiratory tract like: bronchitis, chronic bronchitis, chronic obstructive pulmonary disease (COPD), asthma, bronchiectasis, allergic or non-allergic rhinitis or sinusitis, cystic fibrosis, (1-antitrypsin deficiency, cough, chronic cough, lung emphysema, lung fibrosis and hyperreactive airways, preferably asthma and COPD.

The proportions in which the active substances 1 and 2 may be used in the active substance combinations according to the invention are variable. Active substances 1 and 2 may possibly be present in the form of their solvates or hydrates. Depending on the choice of the compounds 1 and 2, the weight ratios which may be used within the scope of the present invention vary on the basis of the different molecular weights of the various compounds and their different potencies. As a rule, the pharmaceutical combinations according to the invention may contain compounds 1 and 2 in ratios by weight ranging from 1:300 to 50:1, preferably from 1:250 to 40:1. In the particularly preferred pharmaceutical combinations which contain tiotropium salt as compound 1 and 2 the weight ratios are most preferably in a range in which tiotropium 1′ and 2 are present in ratios of 1:150 to 30:1, more preferably from 1:50 to 20:1.

For example, without restricting the scope of the invention thereto, preferred combinations of 1 and andolast 2 according to the invention may contain tiotropium 1′ and 2 in the following proportions by weight: 1:3000, 1:2900, 1:2800,1:2700, 1:2600, 1:2500, 1:2400,1:2300, 1:2200, 1:2100,1:2000, 1:1900, 1:1800, 1:1700, 1:1600, 1:1500, 1:1400, 1:1300, 1:1200, 1:1100, 1:1000, 1:900, 1:800, 1:700, 1:600, 1:500, 1:450, 1:400, 1:1350, 1:300, 1:250, 1:200, 1:150, 1:100, 1:90, 1:80, 1:70, 1:60, 1:50; 1:49; 1:48; 1:47; 1:46; 1:45; 1:44; 1:43; 1:42; 1:41; 1:40; 1:39; 1:38; 1:37; 1:36; 1:35; 1:34; 1:33; 1:32; 1:31; 1:30; 1:29; 1:28; 1:27; 1:26; 1:25; 1:24; 1:23; 1:22; 1:21; 1:20; 1:19; 1:18; 1:17; 1:16; 1:15; 1:14; 1:13; 1:12; 1:11; 1:10; 1:9; 1:8; 1:7; 1:6; 1:5; 1:4; 1:3; 1:2; 1:1; 2:1; 3:1; 4:1; 5:1; 6:1; 7:1; 8:1; 9:1; 10:1; 11:1; 12:1; 13:1; 14:1; 15:1; 16:1; 17:1; 18:1; 19:1; 20:1.

The pharmaceutical compositions according to the invention containing the combinations of 1 and andolast 2 are normally administered so that 1 and andolast 2 are present together in doses of 1 to 10000 μg, preferably from 0.1 to 2000 μg, more preferably from 1 to 1000 μg, even more preferably from 5 to 500 μg, preferably according to the invention from 10 to 300 μg, preferably from 20 to 200 μg per single dose. For example, combinations of 1 and andolast 2 according to the invention contain a quantity of cation 1′ and andolast 2 such that the total dosage per single dose is about 0.1 μg, 0.5 μg, 1 μg, 5 μg, 10 μg, 15 μg, 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg or the like. In these dosage ranges the active substances 1′ and 2 may be present in the weight ratios described above.

For example and without restricting the scope of the invention thereto, the combinations of 1 and andolast 2 according to the invention may contain an amount of cation 1′ and 2 such that in each single dose 1 μg of 1′ and 0.75 μg of 2, 1 μg of 1′ and 1.5 μg of 2, 1 μg of 1′ and 2 μg of 2, 1 μg of 1′ and 4 μg of 2, 1 μg of 1′ and 8 μg of 2, 1 μg of 1′ and 12.5 μg of 2, 1 μg of 1′ and 25 μg of 2, 1 μg of 1′ and 50 μg of 2, 1 μg of 1′ and 100 μg of 2, 1 μg of 1′ and 125 μg of 2, 1 μg of 1′ and 200 μg of 2, 1 μg of 1′ and 250 μg of 2, 5 μg of 1′ and 0.75 μg of 2, 5 μg of 1′ and 1.5 μg of 2, 5 μg of 1′ and 2 μg of 2, 5 μg of 1′ and 4 μg of 2, 5 μg of 1′ and 8 μg of 2, 5 μg of 1′ and 12.5 μg of 2, 5 μg of 1′ and 25 μg of 2, 5 μg of 1′ and 50 μg of 2, 5 μg of 1′ and 100 μg of 2, 5 μg of 1′ and 125 μg of 2, 5 μg of 1′ and 200 μg of 2, 5 μg of 1′ and 250 μg of 2, 10 μg of 1′ and 0.75 μg of 2, 10 μg of 1′ and 1.5 μg of 2, 10 μg of 1′ and 2 μg of 2, 10 μg of 1′ and 4 μg of 2, 10 μg of 1′ and 8 μg of 2, 10 μg of 1′ and 12.5 μg of 2, 10 μg of 1′ and 25 μg of 2, 10 μg of 1′ and 50 μg of 2, 10 μg of 1′ and 100 μg of 2, 10 μg of 1′ and 125 μg of 2, 10 μg of 1′ and 200 μg of 2, 10 μg of 1′ and 250 μg of 2, 18 μg of 1′ and 25 μg of 2, 18 μg of 1′ and 0.75 μg of 2, 18 μg of 1′ and 1.5 μg of 2, 18 μg of 1′ and 2 μg of 2, 18 μg of 1′ and 4 μg of 2, 18 μg of 1′ and 8 μg of 2, 18 μg of 1′ and 12.5 μg of 2, 18 μg of 1′ and 50 μg of 2, 18 μg of 1′ and 100 μg of 2, 18 μg of 1′ and 125 μg of 2, 18 μg of 1′ and 200 μg of 2, 18 μg of 1′ and 250 μg of 2, 20 μg of 1′ and 25 μg of 2, 20 μg of 1′ and 0.75 μg of 2, 20 μg of 1′ and 1.25 μg of 2, 20 μg of 1′ and 2 μg of 2, 20 μg of 1′ and 4 μg of 2, 20 μg of 1′ and 8 μg of 2, 20 μg of 1′ and 12.5 μg of 2, 20 μg of 1′ and 50 μg of 2, 20 μg of 1′ and 100 μg of 2, 20 μg of 1′ and 125 μg of 2, 20 μg of 1′ and 200 μg of 2, 20 μg of 1′ and 250 μg of 2, 36 μg of 1′ and 25 μg of 2, 36 μg of 1′ and 0.75 μg of 2, 36 μg of 1′ and 1.25 μg of 2, 36 μg of 1′ and 2 μg of 2, 36 μg of 1′ and 4 μg of 2, 36 μg of 1′ and 8 μg of 2, 36 μg of 1′ and 12.5 μg of 2, 36 μg of 1′ and 50 μg of 2, 36 μg of 1′ and 100 μg of 2, 36 μg of 1′ and 125 μg of 2, 36 μg of 1′ and 200 μg of 2, 36 μg of 1′ and 250 μg of 2, 40 μg of 1′ and 0.75 μg of 2, 40 μg of 1′ and 1.25 μg of 2, 40 μg of 1′ and 2 μg of 2, 40 μg of 1′ and 4 μg of 2, 40 μg of 1′ and 8 μg of 2, 40 μg of 1′ and 12.5 μg of 2, 40 μg of 1′ and 25 μg of 2, 40 μg of 1′ and 50 μg of 2, 40 μg of 1′ and 100 μg of 2, 40 μg of 1′ and 125 μg of 2, 40 μg of 1′ and 200 μg of 2 or 40 μg of 1′ and 250 μg of 2, 80 μg of 1′ and 0.75 μg of 2, 80 μg of 1′ and 1.25 μg of 2, 80 μg of 1′ and 2 μg of 2, 80 μg of 1′ and 4 μg of 2, 80 μg of 1′ and 8 μg of 2, 80 μg of 1′ and 12.5 μg of 2, 80 μg of 1′ and 25 μg of 2, 80 μg of 1′ and 50 μg of 2, 80 μg of 1′ and 100 μg of 2, 80 μg of 1′ and 125 μg of 2, 80 μg of 1′ and 200 μg of 2 or 80 μg of 1′ and 250 μg of 2, 80 μg of 1′ and 0.75 μg of 2, 80 μg of 1′ and 1.25 μg of 2, 80 μg of 1′ and 2 μg of 2, 80 μg of 1′ and 4 μg of 2, 80 μg of 1′ and 8 μg of 2, 80 μg of 1′ and 12.5 μg of 2, 80 μg of 1′ and 25 μg of 2, 80 μg of 1′ and 50 μg of 2, 80 μg of 1′ and 100 μg of 2, 80 μg of 1′ and 125 μg of 2, 80 μg of 1′ and 200 μg of 2 or 80 μg of 1′ and 250 μg of 2, 120 μg of 1′ and 0.75 μg of 2, 120 μg of 1′ and 1.25 μg of 2, 120 μg of 1′ and 2 μg of 120 μg of 1′ and 4 μg of 2, 120 μg of 1′ and 8 μg of 2, 120 μg of 1′ and 12.5 μg of 2, 120 μg of 1′ and 25 μg of 2, 120 μg of 1′ and 50 μg of 2, 120 μg of 1′ and 100 μg of 2, 120 μg of 1′ and 125 μg of 2, 120 μg of 1′ and 200 μg of 2 or 120 μg of 1′ and 250 μg of 2, 200 μg of 1′ and 0.75 μg of 2, 200 μg of 1′ and 1.25 μg of 2, 200 μg of 1′ and 2 μg of 2, 200 μg of 1′ and 4 μg of 2, 200 μg of 1′ and 8 μg of 2, 200 μg of 1′ and 12.5 μg of 2, 200 μg of 1′ and 25 μg of 2, 200 μg of 1′ and 50 μg of 2, 200 μg of 1′ and 100 μg of 2, 200 μg of 1′ and 125 μg of 2, 200 μg of 1′ and 200 μg of 2 or 200 μg of 1′ and 250 μg of 2, 300 μg of 1′ and 0.75 μg of 2, 300 μg of 1′ and 1.25 μg of 2, 300 μg of 1′ and 2 μg of 2, 300 μg of 1′ and 4 μg of 2, 300 μg of 1′ and 8 μg of 2, 300 μg of 1′ and 12.5 μg of 2, 300 μg of 1′ and 25 μg of 2, 300 μg of 1′ and 50 μg of 2, 300 μg of 1′ and 100 μg of 2, 300 μg of 1′ and 125 μg of 2, 300 μg of 1′ and 200 μg of 2 or 300 μg of 1′ and 250 μg of 2, 600 μg of 1′ and 0.75 μg of 2, 600 μg of 1′ and 1.251 g of 2, 600 μg of 1′ and 2 μg of 2, 600 μg of 1′ and 4 μg of 2, 600 μg of 1′ and 8 μg of 2, 600 μg of 1′ and 12.5 μg of 2, 600 μg of 1′ and 25 μg of 2, 600 μg of 1′ and 50 μg of 2, 600 μg of 1′ and 100 μg of 2, 600 μg of 1′ and 125 μg of 2, 600 μg of 1′ and 200 μg of 2 or 600 μg of 1′ and 250 μg of 2 are administered.

The active substance combinations of 1 and andolast 2 according to the invention are preferably administered by inhalation. For this purpose, ingredients 1 and 2 have to be made available in forms suitable for inhalation. Inhalable preparations include inhalable powders, propellant-containing metered-dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

### A) Inhalable Powder Containing the Combinations of Active Substances 1 and 2 According to the Invention:

The inhalable powders according to the invention may contain 1 and 2 either on their own or in admixture with suitable physiologically acceptable excipients.

If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextran), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 5 μm, is added to the excipient mixture. Processes for producing the inhalable powders according to the invention by grinding and micronising and by finally mixing the ingredients together are known from the prior art. The inhalable powders according to the invention may be prepared and administered either in the form of a single powder mixture which contains both 1 and 2 or in the form of separate inhalable powders which contain only 1 and 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630A, or by other means as described in DE 36 25 685 A. Preferably, the inhalable powders according to the invention which contain physiologically acceptable excipients in addition to 1 and 2 are packed into capsules (to produce so-called inhalettes) which are used in inhalers as described, for example, in WO 94/28958.

A particularly preferred inhaler for using the pharmaceutical combination according to the invention in inhalettes is shown in FIG. 1 of WO 03/084502. This inhaler is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, a mouthpiece 12 which is connected to the housing 1, the deck 3 and a cover 11 via a spindle 10 to enable it to be flipped open or shut and three ho les 13 with diameters below 1 mm in the central region around the capsule chamber 6 and underneath the screen housing 4 and screen 5.

The main air flow enters the inhaler between deck 3 and base 1 near to the hinge. The deck has in this range a reduced width, which forms the entrance slit for the air. Then the flow reverses and enters the capsule chamber 6 through the inlet tube. The flow is then further conducted through the filter and filter holder to the mouthpiece. A small portion of the flow enters the device between mouthpiece and deck and flows then between filterholder and deck into the main stream. Due to production tolerances there is some uncertainty in this flow because of the actual width of the slit between filterholder and deck. In case of new or reworked tools the flow resistance of the inhaler may therefore be a little off the target value. To correct this deviation the deck has in the central region around the capsule chamber 6 and underneath the screen housing 4 and screen 5 three holes 13 with diameters below 1 mm. Through these holes 13 flows air from the base into the main air stream and reduces such slightly the flow resistance of the inhaler. The actual diameter of these holes 13 can be chosen by proper inserts in the tools so that the mean flow resistance can be made equal to the target value.

If the inhalable powders according to the invention are packed into capsules (inhalers) for the preferred use described above, the quantities packed into each capsule should be 1 to 30 mg, preferably 3 to 20 mg, more particularly 5 to 10 mg of inhalable powder per capsule. These capsules contain, according to the invention, either together or separately, the doses of 1′ and 2 mentioned hereinbefore for each single dose.

### B) Propellant Gas-Driven Inhalation Aerosols Containing the Combinations of Active Substances 1 and 2:

Inhalation aerosols containing propellant gas according to the invention may contain substances 1 and 2 dissolved in the propellant gas or in dispersed form. 1 and 2 may be present in separate formulations or in a single preparation, in which 1 and 2 are either each dissolved, dispersed or only one or two of the components is or are dissolved and the other or others is or are dispersed. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG134a (1,1,1,2-tetrafluoroethane) and TG227 (1,1,1,2,3,3,3-heptafluoropropane) and mixtures thereof.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 5 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention. The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

### C) Propellant-Free Inhalable Solutions or Suspensions Containing the Combinations of Active Substances 1 and 2 According to the Invention:

It is particularly preferred to use the active substance combination according to the invention in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include physiologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the required therapeutic dose within a few seconds to produce an aerosol suitable for therapeutic inhalation. Within the scope of the present invention, preferred nebulisers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

This nebuliser (Respimat®) can advantageously be used to produce the inhalable aerosols according to the invention containing the combination of active substances 1 and 2. Because of its cylindrical shape and handy size of less than 9 to 15 cm long and 2 to 4 cm wide, this device can be carried at all times by the patient. The nebuliser sprays a defined volume of pharmaceutical formulation using high pressures through small nozzles so as to produce inhalable aerosols.

The preferred atomiser essentially consists of an upper housing part, a pump housing, a nozzle, a locking mechanism, a spring housing, a spring and a storage container, characterised by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing,
a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 Mpa (about 50 to 600 bar), preferably 10 to 60 Mpa (about 100 to 600 bar) on the fluid, the measured amount of active substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the valve body.

The nozzle in the nozzle body is preferably microstructured, i.e. produced by microtechnology. Microstructured nozzle bodies are disclosed for example in WO-94/07607; reference is hereby made to the contents of this specification, particularly FIG. 1 therein and the associated description.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings. The liquid pharmaceutical preparation strikes the nozzle body with an entry pressure of up to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention.

The atomising process is initiated by pressing gently on the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to which reference is hereby made.

The components of the atomiser (nebuliser) are made of a material which is suitable for its purpose. The housing of the atomiser and, if its operation permits, other parts as well, are preferably made of plastics, e.g. by injection moulding. For medicinal purposes, physiologically safe materials are used.

FIGS. 6a/b of WO 97/12687, which are specifically incorporated by reference into the instant application show the nebuliser (Respimat®) which can advantageously be used for inhaling the aqueous aerosol preparations according to the invention.

FIG. 6a of WO 97/12687 shows a longitudinal section through the atomiser with the spring biased while FIG. 6b of WO 97/12687 shows a longitudinal section through the atomiser with the spring relaxed. The upper housing part (51) contains the pump housing (52) on the end of which is mounted the holder (53) for the atomiser nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomised. The storage container is sealed off by the stopper (73) through which the hollow plunger projects into the storage container and is immersed at its end in the fluid (supply of active substance solution).

The spindle (74) for the mechanical counter is mounted in the covering of the spring housing. At the end of the spindle facing the upper housing part is the drive pinion (75). The slider (76) sits on the spindle.

The nebuliser described above is suitable for nebulising the aerosol preparations according to the invention to produce an aerosol suitable for inhalation.

If the formulation according to the invention is nebulised using the method described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the formulation according to the invention may also be nebulised by means of inhalers other than those described above, e.g. jet stream inhalers or other stationary nebulisers.

Accordingly, in a further aspect, the invention relates to pharmaceutical formulations in the form of propellant-free inhalable solutions or suspensions as described above combined with a device suitable for administering these formulations, preferably in conjunction with the Respimat®. Preferably, the invention relates to propellant-free inhalable solutions or suspensions characterised by the combination of active substances 1 and 2 according to the invention in conjunction with the device known by the name Respimat®. In addition, the present invention relates to the above-mentioned devices for inhalation, preferably the Respimat®, characterised in that they contain the propellant-free inhalable solutions or suspensions according to the invention as described hereinbefore.

The propellant-free inhalable solutions or suspensions according to the invention may take the form of concentrates or sterile inhalable solutions or suspensions ready for use, as well as the above-mentioned solutions and suspensions designed for use in a Respimat®. Formulations ready for use may be produced from the concentrates, for example, by the addition of isotonic saline solutions. Sterile formulations ready for use may be administered using energy-operated fixed or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable nebuliser which produces inhalable aerosols by means of ultrasound or compressed air by the Venturi principle or other methods.

The Examples which follow serve to illustrate the present invention in more detail without restricting the scope of the invention to the following embodiments by way of example.

### EXAMPLES OF FORMULATIONS

Inhalable powders:

| Ingredients | μg per capsule |
|---|---|
| tiotropium bromide monohydrate | 5.125 |
| andolast | 5.000 |
| lactose | 9,989.875 |
| Total | 10,000.00 |
| tiotropium bromide monohydrate | 5.125 |
| andolast | 10,000.00 |
| lactose | 4,989.875 |
| Total | 15,000.00 |
| tiotropium bromide monohydrate | 11.25 |
| andolast | 5,000 |
| lactose | 4,988.75 |
| Total | 10,000.00 |
| tiotropium bromide monohydrate | 22.50 |
| andolast | 5,000 |
| lactose | 4,977.50 |
| Total | 10,000.00 |
| tiotropium bromide monohydrate | 11.25 |
| andolast | 10,000.00 |
| lactose | 4,988.75 |
| Total | 15,000.00 |
| tiotropium bromide monohydrate | 22.50 |
| andolast | 10,000.00 |
| lactose | 4,977.50 |
| Total | 15,000.00 |

1. A pharmaceutical composition, comprising one or more anticholinergics (1) in conjunction with andolast (2), optionally together with a pharmaceutically acceptable excipient.

2. The pharmaceutical composition according to claim 1, wherein the anticholinergic 1 is selected from the group consisting of tiotropium salts, oxitropium salts, ipratropium salts and glycopyrronium salts.

3. The pharmaceutical composition according to claim 1, wherein the anticholinergic 1 is are selected from the group consisting of tropenol 2,2-diphenylpropionic acid ester methobromide, scopine 2,2-diphenylpropionic acid ester methobromide, scopine 2-fluoro-2,2-diphenylacetic acid ester methobromide, tropenol 2-fluoro-2,2-diphenylacetic acid ester methobromide, tropenol 3,3′,4,4′-tetrafluorobenzilic acid ester methobromide, scopine 3,3′,4,4′-tetrafluorobenzilic acid ester methobromide, scopine 4,4′-difluorobenzilic acid ester methobromide, tropenol 4,4′-difluorobenzilic acid ester methobromide, scopine 3,3′-difluorobenzilic acid ester methobromide, tropenol 3,3′-difluorobenzilic acid ester methobromide, tropenol 9-hydroxy-fluorene-9-carboxylate methobromide, tropenol 9-fluoro-fluorene-9-carboxylate methobromide, scopine 9-hydroxy-fluorene-9-carboxylate methobromide, scopine 9-fluoro-fluorene-9-carboxylate methobromide, tropenol 9-methyl-fluorene-9-carboxylate methobromide, scopine 9-methyl-fluorene-9-carboxylate methobromide, cyclopropyltropine benzilate methobromide, cyclopropyltropine 2,2-diphenylpropionate methobromide, cyclopropyltropine 9-hydroxy-xanthene-9-carboxylate methobromide, cyclopropyltropine 9-methyl-fluorene-9-carboxylate methobromide, cyclopropyltropine 9-methyl-xanthene-9-carboxylate methobromide, cyclopropyltropine 9-hydroxy-fluorene-9-carboxylate methobromide, cyclopropyltropine methyl 4,4′-difluorobenzilate methobromide, tropenol 9-hydroxy-xanthene-9-carboxylate methobromide, scopine 9-hydroxy-xanthene-9-carboxylate methobromide, tropenol 9-methyl-xanthene-9-carboxylate methobromide, scopine 9-methyl-xanthene-9-carboxylate methobromide, tropenol 9-ethyl-xanthene-9-carboxylate methobromide, tropenol 9-difluoromethyl-xanthene-9-carboxylate methobromide, scopine 9-hydroxymethyl-xanthene-9-carboxylate methobromide, or corresponding fluorides, chlorides, iodides, sulphates, phosphates, methanesulphonates, nitrates, maleates, acetates, citrates, fumarates, tartrates, oxalates, succinates, benzoates and p-toluenesulphonates thereof.

4. The pharmaceutical composition according to claim 1, wherein the anticholinergic 1 is selected from the compounds of formula 1a 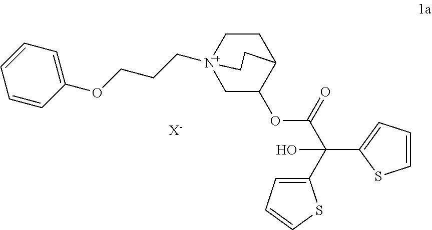
wherein
X⁻ denotes an anion with a single negative charge selected from the group consisting of fluoride, chloride, bromide, iodide, sulphate, phosphate, methanesulphonate, nitrate, maleate, acetate, citrate, fumarate, tartrate, oxalate, succinate, benzoate and p-toluenesulphonate.

5. The pharmaceutical composition according to claim 1, wherein the active substances 1 and 2 are present either together in a single formulation or in two separate formulations.

6. The pharmaceutical composition according to claim 1, wherein 1 contains chloride, bromide, iodide, methanesulphonate, sulphate or paratoluene sulphonate as the counter-ion.

7. The pharmaceutical composition according to claim 1, wherein it is in the form of a formulation suitable for inhalation.

8. The pharmaceutical composition according to claim 7, wherein the formulation is an inhalable powder, propellant-containing metered-dose aerosol or propellant-free inhalable solution or suspension.

9. The pharmaceutical composition according to claim 8, wherein the inhalable powder contains 1 and 2 in admixture with one or more physiologically acceptable excipients selected from the group consisting of monosaccharides, disaccharides, oligo- and polysaccharides, polyalcohols, and salts.

10. The pharmaceutical composition according to claim 8, wherein the propellant-containing inhalable aerosol contains 1 and 2 in dissolved or dispersed form.

11. The pharmaceutical composition according to claim 10, wherein the aerosol contains, as propellant gas, hydrocarbons such as n-propane, n-butane or isobutane or halohydrocarbons such as fluorinated derivatives of methane, ethane, propane, butane, cyclopropane or cyclobutane.

12. The pharmaceutical composition according to claim 11, wherein the propellant gas is TG134a, TG227 or a mixture thereof.

13. The pharmaceutical composition according to claim 8, wherein the propellant-free inhalable solution or suspension contains water, ethanol or a mixture of water and ethanol as solvent.

14. The pharmaceutical composition according to claim 13, wherein the pH is 2-7.

15. A method for treating inflammatory and/or obstructive diseases of the respiratory tract in a patient in need thereof comprising administering to the patient a pharmaceutical composition according to claim 1.

16. The pharmaceutical composition according to claim 1, wherein 1 contains bromide as the counter-ion.

17. The pharmaceutical composition according to claim 14, wherein the pH is 2-5.

18. The method according to claim 15, wherein the inflammatory and/or obstructive disease of the respiratory tract is selected from the group consisting of bronchitis, chronic bronchitis, chronic obstructive pulmonary disease (COPD), asthma, bronchiectasis, allergic or non-allergic rhinitis or sinusitis, cystic fibrosis, α1-antitrypsin deficiency, cough, chronic cough, lung emphysema, lung fibrosis and hyperreactive airways
